(19) United States
(12) Patent Application Publication
(10) Pub. No.: US 2025/0229133 A1
(43) Pub. Date: Jul. 17, 2025

(54) DEVICES, SYSTEMS AND METHODS FOR GENERATING TRAINING PROGRAM RECOMMENDATIONS

(71) Applicant: iFIT Inc, Logan, UT (US)

(72) Inventor: Chase Brammer, Providence, UT (US)

(21) Appl. No.: 19/012,032

(22) Filed: Jan. 7, 2025

Related U.S. Application Data
(60) Provisional application No. 63/621,529, filed on Jan. 16, 2024.

(51) Int. Cl. A63B 24/00; G16H 20/30; G16H 80/00

(52) U.S. Cl. CPC A63B 24/0075; G16H 20/30; G16H 80/00; A63B 2024/0065; A63B 2230/75

(57) ABSTRACT

A training program recommendation system may monitor a user while the user is performing an assessment exercise activity. An exercise chatbot may present the user with an assessment query. The assessment query may request exercise information for the user. A training program recommendation system may apply a health assessment model to the exercise information to determine a health parameter and a user scaling factor. A training program recommendation system may apply a recommendation model to the health parameter and a user response to the assessment query. The recommendation model generates a training program recommendation for the user based at least in part on at least one of the health parameter or the user scaling factor. The training program recommendation includes a training program. A training program recommendation system may provide the training program recommendation to the user.

### CROSS REFERENCE

This application claims the benefit and priority to U.S. Patent Application No. 63/621,529, filed Jan. 16, 2024, which is incorporated herein by reference in its entireties for all that it discloses.

### BACKGROUND

Health is a critical part of a person's well-being. People cultivate their health through health actions, including exercise, diet, lifestyle, and so forth. People may use exercise systems to facilitate their health journey. Conventional exercise systems may provide health recommendations to a person based on pre-determined responses to the person's requests. For example, a user that desires to lose weight and participate in a 5 kilometer race may receive a pre-determined diet and exercise system. But such pre-determined systems are inflexible. Indeed, such plans may be inapplicable to some user's lifestyle, attitude, motivational styles, and so forth.

In some situations, indoor exercise has increased in popularity and accessibility. Many people exercise indoors with the aid of an exercise device. Exercise devices may be designed to simulate outdoor exercise activities, such as a treadmill to simulate running, a stationary bicycle to simulate cycling, or a rower to simulate rowing. Additionally, or alternatively, exercise devices may be designed to exercise a certain muscle or muscle group, reduce the impact or force applied to the user, aid in certain types of indoor exercises, perform any other function, and combinations thereof.

Many exercise devices facilitate playing or implementing an exercise program on the exercise device. The exercise program may include operating information for the exercise device and some interactive features. The interactive features may include videos, including videos of trainers and/or virtual environments. The exercise programs are often stored in an exercise program library. But exercise program libraries may store massive amounts of exercise programs. This may make identifying, searching, and finding exercise programs of interest to a user difficult.

### SUMMARY

In some aspects, the techniques described herein relate to a method for providing training program recommendations. The method includes monitoring a user while the user is performing an assessment exercise activity. The training program recommendation system presents, with an exercise chatbot, the user with an assessment query. The assessment query requests exercise information for the user. The training program recommendation system applies a health assessment model to the exercise information to determine a health parameter and a user scaling factor. The health parameter is based at least in part on the assessment exercise activity. The training program recommendation system applies a recommendation model to the health parameter and a user response to the assessment query. The recommendation model generates a training program recommendation for the user based at least in part on at least one of the health parameter or the user scaling factor. The training program recommendation includes a training program; and providing the training program recommendation to the user.

In some aspects, the techniques described herein relate to a method for providing training program recommendations. The method includes monitoring a user while performing an exercise activity. A training program recommendation system determines a health parameter for the user based on the exercise activity. The training program recommendation system receives exercise information for the user. The training program recommendation system applies a health assessment model to the exercise information and the health parameter. The health assessment model identifies a user scaling factor for the user. The training program recommendation system presents a query to the user. The query requests user information including user goals. The training program recommendation system applies a recommendation model to the user scaling factor and a response from the user including the user goals. The recommendation model generates a training program for the user.

This summary is provided to introduce a selection of concepts that are further described below in the detailed description. This summary is not intended to identify key or essential features of the claimed subject matter, nor is it intended to be used as an aid in limiting the scope of the claimed subject matter. Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a schematic representation of a training program recommendation system, according to at least one embodiment of the present disclosure.

FIG. 2 is a representation of a training program recommendation system, according to at least one embodiment of the present disclosure.

FIG. 3 is a representation of a string diagram, according to at least one embodiment of the present disclosure.

FIG. 4 is a flowchart of a method for generating training program recommendations, according to at least one embodiment of the present disclosure.

FIG. 5 is a flowchart of a method for generating training program recommendations, according to at least one embodiment of the present disclosure.

FIG. 6 is a schematic representation of a computing system, according to at least one embodiment of the present disclosure.

### DETAILED DESCRIPTION

This disclosure generally relates to generating training program recommendations for a user. A training program recommendation system may prepare a recommendation of a training program for a user. A health monitor may monitor a user while the user is performing one or more exercise activities. A health assessment model may determine one or more health parameters for the user based on the monitored properties. An exercise chatbot may present the user with an assessment query. The assessment query may request health information and/or exercise information from the user. The training program recommendation system may apply a recommendation model to the health parameter and the user response (e.g., the health information and/or exercise information). The recommendation model may prepare a recommendation of a training program for the user to perform. The recommendation model may further prepare a user scaling factor. The user scaling factor may be based on the user exercise information and may be used to scale the training programs to the user's capabilities. In this manner, the training program recommendation system may prepare a recommendation of a training program for the user that is tailored to the user, thereby improving compliance and the user's exercise experience.

In accordance with at least one embodiment of the present disclosure, the training program recommendation system may generate a new training program for the user. For example, based on the user's health information and/or exercise information, the training program recommendation system may collect one or more exercise programs that are relevant to the user. The training program recommendation system may identify which exercise programs to perform and in what order (e.g., on what days, what time of day). In some embodiments, the training program recommendation system may generate the training program with multiple types of exercise programs, such as aerobic, anaerobic, indoor exercise, outdoor exercise, group exercise, sports, activities, and so forth. In this manner, the newly generated training program may be tailored to the user's needs.

As illustrated by the foregoing discussion, the present disclosure utilizes a variety of terms to describe features and advantages of the exercise recommendation system. Additional detail is now provided regarding the meaning of such terms. For example, as used herein, the term “exercise information” (e.g., health information) refers to information related to health and/or exercise. In particular, the term exercise information may include information related to one or more exercise activities (e.g., workouts). For example, exercise information may include information related to the performance of the exercise activity, such as exercise activity type, exercise activity day (e.g., date, day of the week), exercise activity time of day, exercise device used, exercise device operating parameters (e.g., resistance, speed, incline level, weight setting), location of exercise device, exercise activity duration, training plan information, any other information related to the performance of the exercise activity, and combinations thereof. In some embodiments, exercise information includes user exercise information. For example, the exercise information may include heartrate information, blood oxygen levels, electrocardiogram (EKG) information, blood sugar information, blood oxygen information, any other user exercise information, and combinations thereof. In some embodiments, exercise information includes user lifestyle or habit information. For example, user lifestyle or habit information may include historical training plan information, sleep information (e.g., duration, time, quality), diet and nutrition information (e.g., food eaten, supplements taken, time of meals), work details, any other user lifestyle or habit information, user exercise goal information, and combinations thereof. In some embodiments, exercise information includes qualitative user exercise information. For example, qualitative user exercise information may include user exercise program ratings, stress levels, pain levels, fatigue levels, attitude levels, motivation levels, any other qualitative user exercise information, and combinations thereof. In some embodiments, health information is information related to a user's health and lifestyle. Exercise information may be information related to the user's exercise activities. In some embodiments, exercise information includes demographic information of the user, including gender, age, altitude, address, any other demographic information, and combinations thereof.

As used herein, a “foundation model” refers to an artificial intelligence (AI) or machine learning (ML) model that is trained to generate an output in response to an input based on a large dataset. The present disclosure may interchangeably refer to foundation models as AI models or ML models. A foundation model may be formed using a neural network having a significant number of parameters (e.g., billions of parameters). The foundation model may utilize the parameters to perform a task or otherwise generate an output based on an input. In one or more embodiments described herein, a foundation model is trained to generate a response to a query. In some implementations, a foundation model refers to a large language model (LLM). The foundation model be trained in any manner. For example, the foundation model may be trained on pattern recognition and text prediction. For example, the foundation model may be trained to predict the next word of a particular sentence or phrase. In one or more implementations described herein, the foundation model refers specifically to an LLM, though other types of foundation models may be used in generating responses to input queries.

As used herein, a chatbot may be a foundation model or an ML model that is trained in natural language algorithms to provide queries to a user and receive responses to the queries. The chatbot may be trained in natural language algorithms that may simulate a human interaction. For example, the chatbot may be trained to generate and present a query using syntax, verbs, nouns, and other grammatical elements to provide information and request information as a human being would. The chatbot may be trained to collect information based on an input dataset, such as the exercise information discussed herein. In some embodiments, the chatbot analyzes the input dataset, identify initial patterns in the input dataset, and request additional information to complete the pattern analysis. In some embodiments, the chatbot receives the pattern analysis from a different ML model, such as an exercise analysis model, health habit model, or other ML model. The chatbot may be interactive. For example, the chatbot may be trained to analyze the received response and generate additional content to provide the user. Such content may include recommendations, additional queries, motivational information, any other content, and combinations thereof.

As used herein, a recommendation model may refer to a foundation model that is trained to generate training program recommendations based on an input dataset. The input dataset may include exercise information and/or historical exercise information. Historical exercise information may include any exercise information previously collected. In some embodiments, historical exercise information includes exercise information related to exercise activities prior to the most recent exercise activities. In some embodiments, historical exercise information includes daily exercise information for a period of time (e.g., one day, one week, one month, one year, multiple years). The recommendation model may be trained on a recommendation training dataset. The recommendation training dataset may include exercise information from people and/or prior users that exhibit positive exercise habits and/or have met previously set exercise goals or health habit goals. The recommendation model may receive the exercise information for a user and compare the user's exercise information, exercise goals, and health habit goals to the patterns identified by the recommendation model. The recommendation model may provide behavioral changes for the user to implement to meet his or her goals.

As used herein, the term “health habits” may refer to actions that are consistently performed that result in outcomes associated with positive health. Health habits may refer to any type of behavior, such as sleep-related actions, exercise activity-related actions, food and meal-related actions, any other health actions, and combinations thereof. In some embodiments, health habits are different for different people. In some embodiments, the recommendation model identifies commonalities in the exercise information and health habits, and provide recommendations to change the health habits.

As used herein, an exercise recommendation may be used to refer to any recommendation to improve a user's health, including a recommendation to improve health habits and/or exercise consistency by implementing a training program. For example, the exercise recommendation may include a change in behavior that may be associated with a user's health habits.

As used herein, a training program may include a series of one or more training activities. The training activities may be any type of activity related to a user's health. For example, training activities may include exercise activities, exercise programs, exercises, stretches, health activities, meals, snacks, dietary guidelines, any other training activities, and combinations thereof. In some embodiments, a training program includes certain training activities that are to be performed in a particular order (e.g., the training activities are arranged in a training program order). In some embodiments, the training program includes training activities that are to be performed in a particular time. In some embodiments, the training program includes training activities that are to be performed within a particular timeframe or within a proximity with another training activity. In some embodiments, the training program includes training activities designed to generate and/or improve a user's health habits.

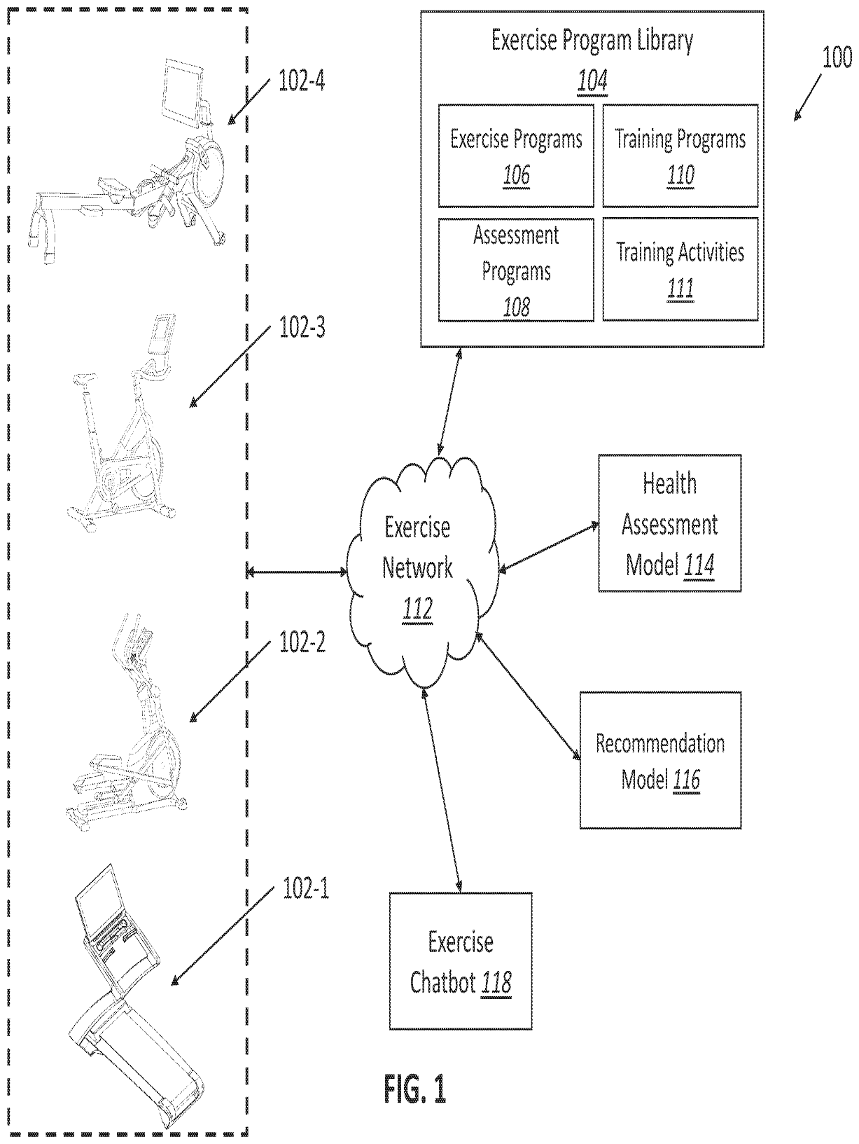

FIG. 1 is a schematic representation of a training program recommendation system 100, according to at least one embodiment of the present disclosure. As discussed herein, the training program recommendation system 100 may provide exercise program recommendations to a user using an ML personalization model applied to exercise programs that have been vectorized by a vector search model.

The training program recommendation system 100 may include one or more exercise devices (collectively 102). The exercise devices 102 may include any type of exercise device, such as a treadmill 102-1, an elliptical device 102-2, a stationary bicycle 102-3, a rower 102-4, a cable extension device, a mirror including a backlit display behind a mirrored surface, any other exercise device, and combinations thereof. While specific exercise devices 102 are illustrated and discussed herein, it should be understood that the techniques of the present disclosure may be applied to any exercise device that is capable of implementing an exercise program.

The training program recommendation system 100 may further include an exercise program library 104. The exercise program library 104 may include a repository of one or more exercise programs 106, assessment programs 108, training programs 110, training activities 111, and other exercise-related information or health-related information. Various elements from the exercise program library 104 may be implemented on one or more of the exercise devices 102, such as the exercise programs 106, the assessment programs 108, the training programs 110, the training activities 111, and combinations thereof.

When the user accesses the exercise program 106 (or other activity from the exercise program library 104) on an exercise device 102, the exercise program 106 may guide the user through a workout. Throughout the workout, the exercise device controls may adjust one or more operating parameters of the exercise device 102 of an operating feature of the exercise device 102. The exercise device controls may adjust any operating parameter of the operating feature, such as a flywheel resistance, a belt speed, a device incline, any other operating parameter, and combinations thereof. In some embodiments, the exercise device controls include a duration for one or more portions of the exercise program. For example, the exercise device controls may include a total workout duration for the entire exercise program 106 and/or an interval duration for a period of a particular difficulty level (e.g., resistance level, belt speed, incline level).

In some embodiments, the training program recommendation system 100 collects exercise information from other sources. For example, the training program recommendation system 100 may collect exercise information from one or more wearable devices, such as a smartwatch, a smart ring, a sleep monitor, a heartrate monitor, any other type of wearable device, and combinations thereof. In some examples, the training program recommendation system 100 may collect the exercise information from a computing device, such as a mobile device, a smartphone, a tablet, a laptop computer, a desktop computer, a server computer, any other computing device, and combinations thereof. In some embodiments, the training program recommendation system 100 may collect exercise information using any other type of user device, such as medical devices, GPS trackers, pedometers, any other user devices, and combinations thereof.

The exercise recommendation system 100 may share the exercise information with one or more ML models, such as a health assessment model 114, a health habit model, a recommendation model 116, and an exercise chatbot 118. The ML models may analyze the exercise information to provide recommendations to the user. For example, the health assessment model 114 may analyze the exercise information and identify one or more health parameters for the user. To identify the health parameters, the health assessment model 114 may analyze the exercise information and determine patterns in the user's behavior, including exercise performance (e.g., strength, speed, coordination), health habits, diet information, any other health parameter, and combinations thereof.

The exercise recommendation system 100 may apply the recommendation model 116 to generate training program recommendations. For example, the recommendation model 116 may receive the exercise information, the health parameter and other information to prepare or generate one or more recommendations for the user to implement or change a behavior to improve the user's health and/or exercise consistency. This may help the user to make and attain his or her health and exercise goals.

In accordance with at least one embodiment of the present disclosure, the exercise chatbot 118 may provide the user with an assessment query. The assessment query may request exercise information from the user. In some embodiments, the assessment query requests other information from the user, including exercise goals, weight loss goals, health goals, interests, experience, work, lifestyle, activity level, any other information, and combinations thereof.

In some embodiments, the exercise chatbot 118 enters into a conversation with the user. For example, the exercise chatbot 118 may receive the response including the additional exercise information from the user. The health assessment model 114 and/or the recommendation model 116 may analyze the received additional exercise information. Based on the analysis of the additional exercise information, the exercise chatbot 118 may generate a follow-up query for the user asking for further exercise information. The follow-up query may be related to the received additional exercise information provided by the user as the response to the first query.

The exercise chatbot 118 may send the follow-up query to the user, and the user may provide follow-up exercise information based on the follow-up query. In this manner, the user may enter into a chat or a conversation with the user. The exercise chatbot 118 may provide any number of follow-up queries to the user, and the user may provide any number of responses. In this manner, the exercise chatbot 118 may receive more exercise information with which the training program recommendation system 100 may prepare training program recommendations.

In some embodiments, the follow-up query includes a response to a question from the user. For example, in the response to one or more of the queries, the user may provide a question to the exercise chatbot 118. The question may be any type of question, such as a request for additional information, a request for clarification, a health question related to health information, an exercise information question related to a particular piece of exercise information or set of exercise information, an exercise activity question related to a particular exercise activity, an exercise device question related to a particular exercise device, any other request for information, and combinations thereof.

In some embodiments, the follow-up query is processed or generated using natural-language processing. For example, the exercise chatbot 118 may include an ML model trained to generate natural language responses. This may help to improve the responsiveness of the exercise chatbot 118 and/or improve the engagement of the user with the exercise chatbot 118.

Using the received exercise information, the recommendation model 116 may prepare a training program recommendation for the user. The training program recommendation may include any recommendation. For example, the training program recommendation may include a recommendation that the user implements one of the pre-prepared training programs 110 from the exercise program library 104. In some examples, the training program recommendation may include a recommendation that the user perform a particular exercise program 106 and/or training activity 111. In some examples, the training program recommendation may include a request that the user perform an assessment program 108. In some examples, the training program recommendation may request that the user perform the assessment program 108 when the user has not previously performed an assessment program 108. In some examples, the training program recommendation may request that the user perform the assessment program 108 when the user has previously completed an assessment program 108, such as to collect additional assessment information and/or to provide a progress update on the results of the user implementing a training program.

In some embodiments, the health assessment model 114 may utilize exercise information collected from the assessment program(s) 108 to generate the health parameter of the user. For example, the health parameter may include a VO2 max. The health assessment model 114 may calculate the VO2 max using exercise information collected during execution of the assessment program 108. In some embodiments, the health parameter may include any other health parameter, such as average pace, weights lifted, repetitions of an activity, speed, any other health parameter, and combinations thereof.

In some embodiments, the recommendation model 116 may generate the training program recommendation based on the health parameter and/or based on the exercise information collected during the assessment program 108. In some embodiments, the recommendation model 116 may generate the training program recommendation using historical exercise information and/or health information. In some embodiments, the recommendation model 116 may generate the training program recommendation using the responses to the assessment query asked by the exercise chatbot 118.

In some embodiments, the recommendation model 116 may generate a training program recommendation that the user implement one or more of the training programs 110 stored in the exercise program library 104. In some embodiments, the recommendation model 116 may generate a new training program recommendation using one or more of the exercise programs 106, the training activities 111, the assessment program 108, portions of the training programs 110, complete training programs 110 included within the new training program recommendation, exercise activities, any other activity, and combinations thereof. The newly generated training program may be tailored to the user. This may help to improve the user's exercise experience.

In some embodiments, the recommendation model 116 may prepare an adjusted or an amended training program recommendation while the user is implementing a training program (including while the user is implementing a training program previously recommended by the training program recommendation system 100). For example, the recommendation model 116 may receive exercise information from the user when the user performs a training activity 111 that is part of the training program. The health assessment model 114 may assess whether the user's health parameter has changed. If the user's health parameter has changed, then the recommendation model 116 may generate an adjusted training program recommendation. For example, if the user's VO2 max has increased, then the recommendation model 116 may provide a recommendation for an increased intensity of the activities included in the training program.

In some embodiments, the health assessment model 114 may determine that the user's exercise status or health parameter have been changed based on the user's performance of a mid-training program assessment program 108. In some embodiments, the mid-training program assessment program 108 is included as part of the training program. In some embodiments, the mid-training program assessment program 108 is performed by the user when the user thinks he or she has improved their exercise parameter. In some embodiments, the mid-training program assessment program 108 is performed by the user when a trainer provides the recommendation. In some embodiments, the mid-training program assessment program 108 is performed by the user when the health assessment model 114 determines that the user's health parameter has changed.

In accordance with at least one embodiment of the present disclosure, the training program recommendation system 100 may generate a scaling factor for the user. The scaling factor may be used to scale an intensity of activities in the exercise program performed by the user. For example, an exercise programs 106 included in the training program 110 may be performed by and/or based on a performance by a professional athlete. Many users may not be able to perform at the same level of intensity (e.g., strength level, speed, pace, weight setting, resistance setting, number of repetitions, exercise form (e.g., motions)) as the professional athlete. The training program recommendation system 100 may generate a user scaling factor for the user that may adjust the exercise program 106 to the user's intensity level based on the user scaling factor. In this manner, the user may be able to perform the same or similar workouts as the professional athlete.

The health assessment model 114 may identify a user intensity based on the assessment program 108 and/or the user's exercise information. The user intensity may be based on any health parameter. For example, the user intensity may be based on VO2 max, speed, pace, strength, experience, exercise activity form, any other health parameter, and combinations thereof.

Based, at least in part, on the user intensity, the health assessment model 114 and/or the recommendation model 116 may generate the user scaling factor. The user scaling factor may be any type of factor. For example, the user scaling factor may be a percentage or a ratio of the user's anticipated performance compared to a baseline intensity. The exercise programs 106 and/or the training activities 111 may have an activity performance level that is compared to the same baseline intensity. Based on the difference between the user scaling factor and the activity performance level, the exercise program may be scaled to the appropriate level of intensity for the user. In some embodiments, the baseline intensity is different for at least two of the exercise activities.

The scaling factor may scale different activities differently. For example, different trainers may perform the same activity at different levels. To maintain the appropriate level of intensity for the user, the user scaling factor may scale the same activity to the same level for the user, regardless of the trainer or the initial activity level. This may help to maintain a seamless transition between activities for the user.

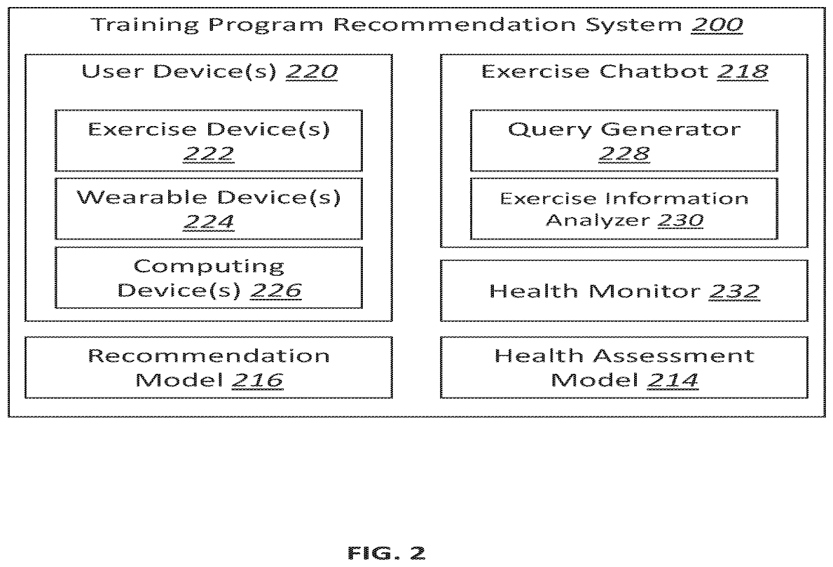

FIG. 2 is a representation of a training program recommendation system 200, according to at least one embodiment of the present disclosure. Each of the components of the training program recommendation system 200 can include software, hardware, or both. For example, the components can include one or more instructions stored on a computer-readable storage medium and executable by processors of one or more computing devices, such as a client device or server device. When executed by the one or more processors, the computer-executable instructions of the training program recommendation system 200 can cause the computing device(s) to perform the methods described herein. Alternatively, the components can include hardware, such as a special-purpose processing device to perform a certain function or group of functions. Alternatively, the components of the training program recommendation system 200 can include a combination of computer-executable instructions and hardware.

Furthermore, the components of the training program recommendation system 200 may, for example, be implemented as one or more operating systems, as one or more stand-alone applications, as one or more modules of an application, as one or more plug-ins, as one or more library functions or functions that may be called by other applications, and/or as a cloud-computing model. Thus, the components may be implemented as a stand-alone application, such as a desktop or mobile application. Furthermore, the components may be implemented as one or more web-based applications hosted on a remote server. The components may also be implemented in a suite of mobile device applications or “apps.”

The exercise recommendation system 200 may include one or more user devices 220. The user may interact with the user devices 220 to communicate with the exercise recommendation system 200. For example, the user may input exercise information into the user devices 220. In some examples, the user may use the user devices 220 to collect exercise information. The user devices 220 may include one or more sensors, and the sensors on the user devices 220 may collect exercise information about the user. As discussed herein, the user devices 220 may include any type of user device, including exercise devices 222, wearable devices 224, computing devices 226, any other user device 220, and combinations thereof.

The exercise recommendation system 200 may further include an exercise chatbot 228. The exercise chatbot 228 may communicate with the user regarding exercise information. For example, the exercise chatbot 228 may present the user with queries. The user may respond to the queries to the exercise chatbot 228. As discussed herein, the exercise chatbot 228 may provide multiple queries to the user and the user may respond to the multiple queries. In this manner, the exercise chatbot 228 may hold a conversation or other back-and-forth engagement with the user.

The exercise chatbot 218 may include a query generator. The query generator may be trained to generate queries for the user. For example, the query generator may be trained in natural-language processing to provide queries to the user in a manner that the user may easily understand and be comfortable engaging in. In some examples, the query generator may be trained on exercise information, as discussed herein.

The exercise chatbot 218 may further include an exercise information analyzer 230. The exercise information analyzer 230 may analyze exercise information, including exercise information received by the user. The exercise information analyzer 230 may communicate with the query generator to prepare the queries.

The training program recommendation system 200 may include a health monitor 232. The health monitor 232 may receive the exercise information for the user, including exercise information from the user devices 220 and/or the exercise chatbot 218. In some embodiments, the health monitor 232 receives the exercise information collected from the user during the health assessment activity. The health monitor 232 may collect and organize the exercise information and/or health information from the user.

The training program recommendation system 200 may further include a health assessment model 214. The health assessment model 214 may analyze the exercise information to generate one or more health parameters for the user. In some embodiments, the health assessment model 214 may generate a user scaling factor for the user.

The exercise recommendation system 200 may further include a recommendation model 216. The exercise recommendation system 200 may apply the recommendation model 216 to the exercise information. For example, the recommendation model 216 may receive the exercise information from the user devices 220 and/or additional exercise information from the exercise chatbot 218. In some embodiments, the recommendation model 216 receives exercise analyses from the health assessment model 214. The recommendation model 216 may be trained to generate a training program recommendation for the user. The recommendation model 216 may provide the exercise recommendation to the user devices 202. In some examples, the recommendation model 216 may provide the training program recommendation to the exercise chatbot 218 and the exercise chatbot 218 may provide the training program recommendation to the user as a query and/or as part of a conversation with the user.

As discussed herein, the recommendation model 216 may generate the training program recommendation for the user. The training program recommendation may include any training program, including a sequence of exercise activities, training activities, dietary guidelines, health habits, any other activity, and combinations thereof.

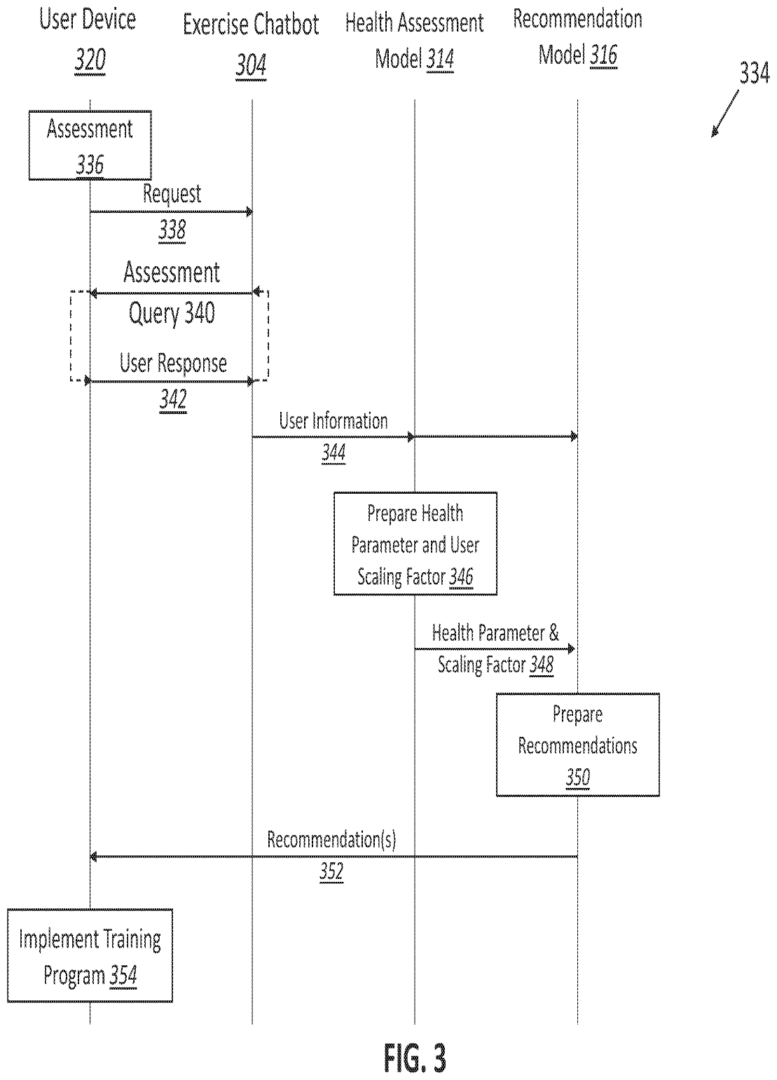

FIG. 3 is a representation of a string diagram 334, according to at least one embodiment of the present disclosure. A user may perform an assessment 336 on a user device 320. The assessment 336 may be any type of assessment, such as an assessment exercise activity including pre-determined assessment exercise activities, conventional exercise activities from which assessment exercise information may be obtained, an assessment quiz, any other assessment information, and combinations thereof.

In some embodiments, the user sends, from the user device 320, a request 338 for an exercise training program. In some embodiments, the request 338 is a user-initiated request. For example, the user may provide an input requesting an exercise training program. In some embodiments, the request 338 is a system-initiated request. For example, the training program recommendation system may identify that the user has performed a particular exercise activity and/or a particular type of exercise activity. The training program recommendation system may generate the request 338 based on the previously performed exercise activity. In some examples, the training program recommendation system may generate the request 338 based on other information, such as the user's exercise information, the user's social media, the user's messages to others, any other information, and combinations thereof.

In some embodiments, an exercise chatbot 304 may receive exercise information from the user. For example, the exercise chatbot 304 may receive exercise information from the assessment 336, the request 338, any other exercise information about the user, and combinations thereof. The exercise chatbot 304 may generate an assessment query 340. The assessment query 340 may include a request for additional information from the user. The assessment query 340 may include a request for any type of information. For example, the assessment query 340 may include a request for the user's goals, fitness level, health habits, diet, interests, any other user information, and combinations thereof.

The exercise chatbot 304 may send the assessment query 340 to the user. For example, the exercise chatbot 304 may send the assessment query 340 to the user device 320. The user may receive the assessment query 340 at the user device 320 and generate a user response 342. The user response 342 may include information that is responsive to the assessment query 340. For example, the user response 342 may include exercise information, goal information, health information, diet information, interests, fitness level, any other user information, and combinations thereof.

As discussed herein, the exercise chatbot 304 may enter into a conversation with the user. For example, the exercise chatbot 304 may receive the user response 342, including the user information in the user response 342, and prepare additional assessment queries 340 or follow-up assessment queries 340. The additional assessment queries 340 may include any request. For example, an additional assessment query 340 may include a request for additional information. In some examples, an additional assessment query 340 may include a request for clarification of the user response 342. In some examples, the additional assessment query 340 may be part of a series of queries the exercise chatbot 304 may utilize to request information from the user. The user may provide additional user responses 342 to the additional assessment queries 340. This process may be repeated until the exercise chatbot 304 has requested all the desired user information and/or until the user has provided all the requested user information.

The exercise chatbot 304 may provide the received user information 344 to other elements of the training program recommendation system. For example, the exercise chatbot 304 may provide the user information 344 to a health assessment model 314 and/or a recommendation model 316. In some examples, the exercise chatbot 304 may instruct the user device 320 to provide the user information 344 to the health assessment model 314 and the recommendation model 316.

The health assessment model 314 may receive the user information and prepare 346 health a health parameter for the user. As discussed herein, the health parameter may be any health parameter used to assess a user's health. For example, the health parameter may include the users VO2 max. The health assessment model 314 may further prepare 346 a user scaling factor. The user scaling factor may be a representation of the user's exercise ability and/or intensity. In some embodiments, the user scaling factor is normalized to a particular normalizing scale. For example, the user scaling factor may be normalized to a particular exercise parameter, such as speed, pace, strength, resistance level, any other exercise parameter, and combinations thereof. In some examples, the user scaling factor may be normalized to a particular intensity level. In some examples, the user scaling factor may be normalized to a trainer's fitness level. For example, the user's scaling factor may be normalized based on a ratio between the user's VO2 max and a trainer VO2 max of a trainer in the training program.

In some embodiments, the health assessment model 314 prepares 346 a single user scaling factor for the user. The single user scaling factor may be used to scale each of the exercise activities in a particular training plan. In some embodiments, the health assessment model 314 prepares 346 multiple user scaling factors for the user. In this manner, at least two of the exercise activities may have different user scaling factors. For example, the user may have different fitness or intensity levels for different activities. As a specific, non-limiting example, a user may have a relatively high intensity level for running and a relatively low intensity level for weight lifting. The health assessment model 314 may prepare 346 different scaling factors for different activities and/or activity types. This may help to adjust each exercise activity to the user's ability and/or intensity, thereby improving the user experience.

The health assessment model 314 may send the health parameter and scaling factor 348 to the recommendation model 316. The recommendation model 316 may utilize the health parameter, the scaling factor, and the user information 344 to prepare 350 one or more training program recommendations. For example, the recommendation model 316 may prepare a recommendation of a pre-existing training program. In some examples, the recommendation model 316 may prepare a recommendation for a new training program using pre-existing exercise activities and other training activities.

The recommendation model 316 may send the training program recommendations 352 to the user. In some embodiments, the recommendation model 316 sends a single training program recommendation 352 to the user. This may help to reduce the user's confusion and stress in selecting a training program recommendation. In some embodiments, the recommendation model 316 sends multiple training program recommendations 352 to the user. This may allow the user to select which training program he or she favors.

In accordance with at least one embodiment of the present disclosure, when the user selects the training program, the user device 320 may implement 354 the training program. For example, the user device 320 may implement, in the sequence of the training program, the training activities from the training program. In some embodiments, a particular user device 320 may implement the training activities that are capable of being implement on the user device 320. For example, a treadmill may implement training activities involving walking or running. In some embodiments, the user device 320 includes multiple user devices, with each user device implementing training activities that are capable of being implemented on the user device 320. In this manner, the training program may help to improve the user's health and/or compliance with a particular training program.

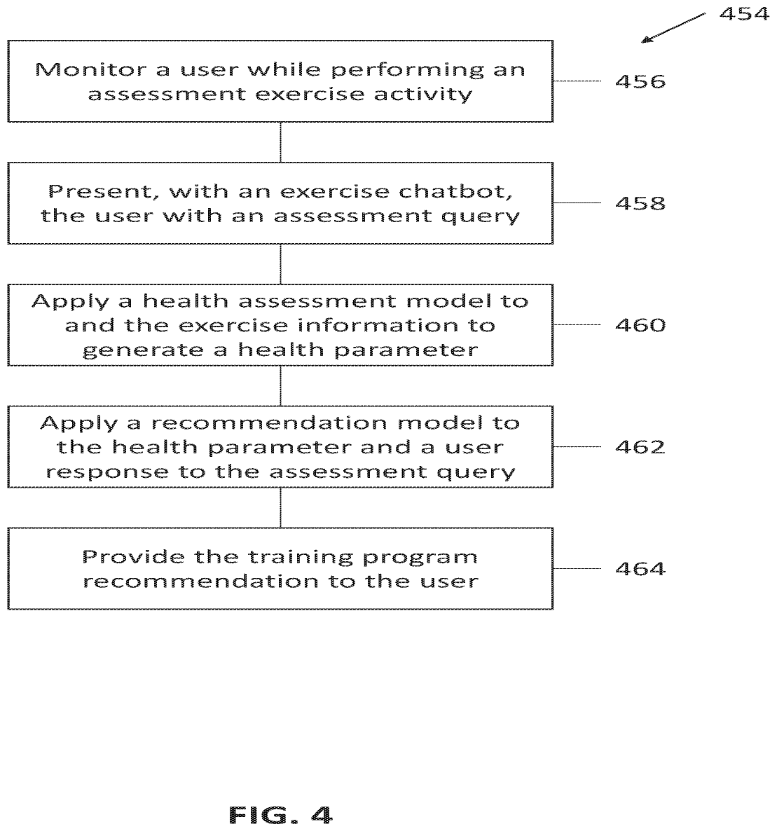

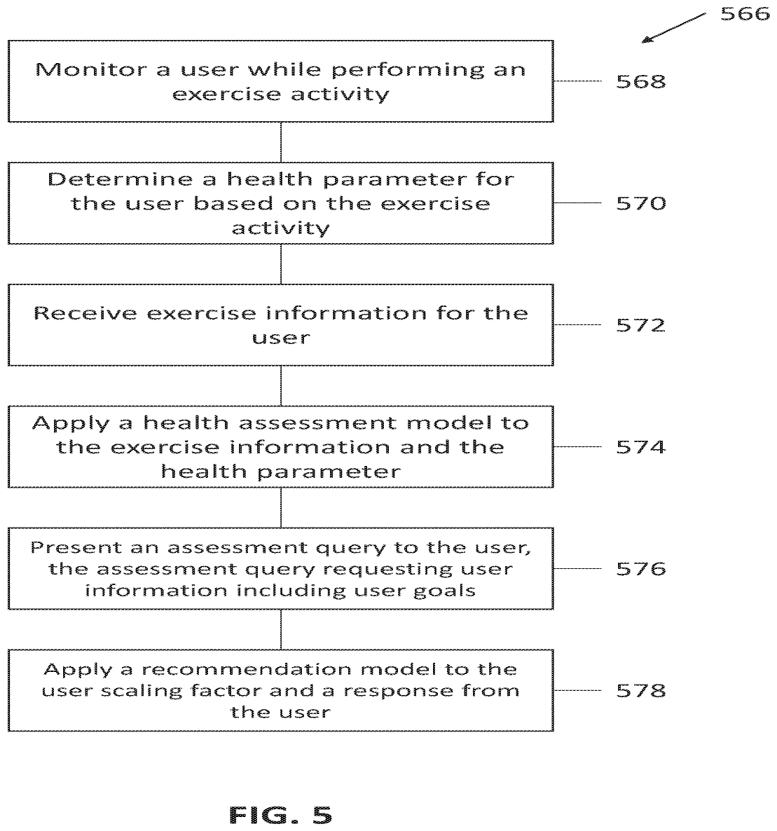

FIGS. 4 and 5, the corresponding text, and the examples provide a number of different methods, systems, devices, and computer-readable media of the training program recommendation system 200. In addition to the foregoing, one or more embodiments can also be described in terms of flowcharts comprising acts for accomplishing a particular result, as shown in FIGS. 4 and 5. FIGS. 4 and 5 may be performed with more or fewer acts. Further, the acts may be performed in differing orders. Additionally, the acts described herein may be repeated or performed in parallel with one another or parallel with different instances of the same or similar acts.

As mentioned, FIG. 4 illustrates a flowchart of a series of acts or a method 454 for generating exercise program recommendations, according to at least one embodiment of the present disclosure. While FIG. 4 illustrates acts according to one embodiment, alternative embodiments may omit, add to, reorder, and/or modify any of the acts shown in FIG. 4. The acts of FIG. 4 can be performed as part of a method. Alternatively, a computer-readable medium can comprise instructions that, when executed by one or more processors, cause a computing device to perform the acts of FIG. 4. In some embodiments, a system can perform the acts of FIG. 4.

In accordance with at least one embodiment of the present disclosure, the training program recommendation system may monitor a user while the user is performing an assessment exercise activity at 456. As discussed herein, the assessment exercise activity may be any exercise activity, including an exercise specifically directed to assessing the user's fitness or intensity level, a previously performed exercise activity, a regularly scheduled exercise activity, any other exercise activity, and combinations thereof.

In some embodiments, the training program recommendation system presents, with an exercise chatbot, the user with an assessment query at 458. The training program recommendation system may apply a health assessment model to the exercise information to generate a health parameter at 460. The health parameter may be based, at least in part, on the assessment exercise activity. In some embodiments, the training program recommendation system may determine a user scaling factor based on the assessment exercise activity and/or the user's responses to the assessment query.

The training program recommendation system may apply a recommendation model to the health parameter and the user's response to the assessment query at 462. The recommendation model may generate a training program recommendation for the user. In some embodiments, the training program recommendation is based on the health parameter and/or the user scaling factor. The training program recommendation may include a training program. The training program recommendation system may provide the training program to the user at 464.

As mentioned, FIG. 5 illustrates a flowchart of a series of acts or a method 566 for generating exercise program recommendations, according to at least one embodiment of the present disclosure. While FIG. 5 illustrates acts according to one embodiment, alternative embodiments may omit, add to, reorder, and/or modify any of the acts shown in FIG. 5. The acts of FIG. 5 can be performed as part of a method. Alternatively, a computer-readable medium can comprise instructions that, when executed by one or more processors, cause a computing device to perform the acts of FIG. 5. In some embodiments, a system can perform the acts of FIG. 5.

The training program recommendation system may monitor a user while performing an exercise activity at 568. As discussed herein, the exercise activity may be any exercise activity, including an assessment exercise activity and/or an exercise activity performed without the intent to receive an assessment. The training program recommendation system may determine a health parameter for the user based on the exercise activity at 570. The training program recommendation system may receive exercise information for the user at 572.

The training program recommendation system may apply a health assessment model to the exercise information and the health parameter at 574. The heal assessment model may identify a user scaling factor for the user. The training program recommendation system may present an assessment query to the user at 576. The assessment query may request user information. The requested user information may include user goals. The training program recommendation system may apply a recommendation model to the user scaling factor and a response from the user at 578. The response from the user may include the user goals. The recommendation model may generate a training program for the user. The training program may be scaled based on the user scaling factor.

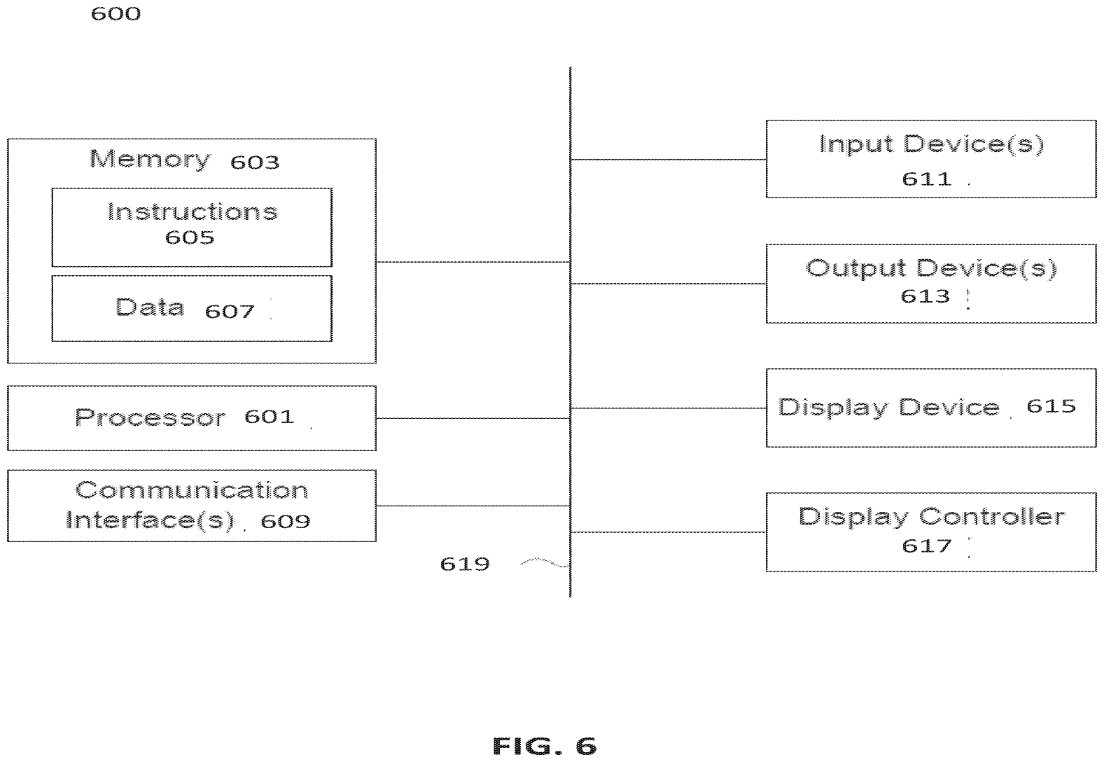

FIG. 6 illustrates certain components that may be included within a computer system 600. One or more computer systems 600 may be used to implement the various devices, components, and systems described herein.

The computer system 600 includes a processor 601. The processor 601 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 601 may be referred to as a central processing unit (CPU). Although just a single processor 601 is shown in the computer system 600 of FIG. 6, in an alternative configuration, a combination of processors (e.g., an ARM and DSP) could be used.

The computer system 600 also includes memory 603 in electronic communication with the processor 601. The memory 603 may be any electronic component capable of storing electronic information. For example, the memory 603 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 605 and data 607 may be stored in the memory 603. The instructions 605 may be executable by the processor 601 to implement some or all of the functionality disclosed herein. Executing the instructions 605 may involve the use of the data 607 that is stored in the memory 603. Any of the various examples of modules and components described herein may be implemented, partially or wholly, as instructions 605 stored in memory 603 and executed by the processor 601. Any of the various examples of data described herein may be among the data 607 that is stored in memory 603 and used during execution of the instructions 605 by the processor 601.

A computer system 600 may also include one or more communication interfaces 609 for communicating with other electronic devices. The communication interface(s) 609 may be based on wired communication technology, wireless communication technology, or both. Some examples of communication interfaces 609 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 600 may also include one or more input devices 611 and one or more output devices 613. Some examples of input devices 611 include a keyboard, mouse, microphone, remote control device, button, joystick, trackball, touchpad, and lightpen. Some examples of output devices 613 include a speaker and a printer. One specific type of output device that is typically included in a computer system 600 is a display device 615. Display devices 615 used with embodiments disclosed herein may utilize any suitable image projection technology, such as liquid crystal display (LCD), light-emitting diode (LED), gas plasma, electroluminescence, or the like. A display controller 617 may also be provided, for converting data 607 stored in the memory 603 into text, graphics, and/or moving images (as appropriate) shown on the display device 615.

The various components of the computer system 600 may be coupled together by one or more buses, which may include a power bus, a control signal bus, a status signal bus, a data bus, etc. For the sake of clarity, the various buses are illustrated in FIG. 6 as a bus system 619.

### INDUSTRIAL APPLICABILITY

This disclosure generally relates to generating training program recommendations for a user. A training program recommendation system may prepare a recommendation of a training program for a user. A health monitor may monitor a user while the user is performing one or more exercise activities. A health assessment model may determine one or more health parameters for the user based on the monitored properties. An exercise chatbot may present the user with an assessment query. The assessment query may request health information and/or exercise information from the user. The training program recommendation system may apply a recommendation model to the health parameter and the user response (e.g., the health information and/or exercise information). The recommendation model may prepare a recommendation of a training program for the user to perform. The recommendation model may further prepare a user scaling factor. The user scaling factor may be based on the user exercise information and may be used to scale the training programs to the user's capabilities. In this manner, the training program recommendation system may prepare a recommendation of a training program for the user that is tailored to the user, thereby improving compliance and the user's exercise experience.

In accordance with at least one embodiment of the present disclosure, the training program recommendation system may generate a new training program for the user. For example, based on the user's health information and/or exercise information, the training program recommendation system may collect one or more exercise programs that are relevant to the user. The training program recommendation system may identify which exercise programs to perform and in what order (e.g., on what days, what time of day). In some embodiments, the training program recommendation system may generate the training program with multiple types of exercise programs, such as aerobic, anaerobic, indoor exercise, outdoor exercise, group exercise, sports, activities, and so forth. In this manner, the newly generated training program may be tailored to the user's needs.

As illustrated by the foregoing discussion, the present disclosure utilizes a variety of terms to describe features and advantages of the exercise recommendation system. Additional detail is now provided regarding the meaning of such terms. For example, as used herein, the term “exercise information” (e.g., health information) refers to information related to health and/or exercise. In particular, the term exercise information may include information related to one or more exercise activities (e.g., workouts). For example, exercise information may include information related to the performance of the exercise activity, such as exercise activity type, exercise activity day (e.g., date, day of the week), exercise activity time of day, exercise device used, exercise device operating parameters (e.g., resistance, speed, incline level, weight setting), location of exercise device, exercise activity duration, training plan information, any other information related to the performance of the exercise activity, and combinations thereof. In some embodiments, exercise information includes user exercise information. For example, the exercise information may include heartrate information, blood oxygen levels, electrocardiogram (EKG) information, blood sugar information, blood oxygen information, any other user exercise information, and combinations thereof. In some embodiments, exercise information includes user lifestyle or habit information. For example, user lifestyle or habit information may include historical training plan information, sleep information (e.g., duration, time, quality), diet and nutrition information (e.g., food eaten, supplements taken, time of meals), work details, any other user lifestyle or habit information, user exercise goal information, and combinations thereof. In some embodiments, exercise information includes qualitative user exercise information. For example, qualitative user exercise information may include user exercise program ratings, stress levels, pain levels, fatigue levels, attitude levels, motivation levels, any other qualitative user exercise information, and combinations thereof. In some embodiments, health information is information related to a user's health and lifestyle. Exercise information may be information related to the user's exercise activities. In some embodiments, exercise information includes demographic information of the user, including gender, age, altitude, address, any other demographic information, and combinations thereof.

As used herein, a “foundation model” refers to an artificial intelligence (AI) or machine learning (ML) model that is trained to generate an output in response to an input based on a large dataset. The present disclosure may interchangeably refer to foundation models as AI models or ML models. A foundation model may be formed using a neural network having a significant number of parameters (e.g., billions of parameters). The foundation model may utilize the parameters to perform a task or otherwise generate an output based on an input. In one or more embodiments described herein, a foundation model is trained to generate a response to a query. In some implementations, a foundation model refers to a large language model (LLM). The foundation model be trained in any manner. For example, the foundation model may be trained on pattern recognition and text prediction. For example, the foundation model may be trained to predict the next word of a particular sentence or phrase. In one or more implementations described herein, the foundation model refers specifically to an LLM, though other types of foundation models may be used in generating responses to input queries.

As used herein, a chatbot may be a foundation model or an ML model that is trained in natural language algorithms to provide queries to a user and receive responses to the queries. The chatbot may be trained in natural language algorithms that may simulate a human interaction. For example, the chatbot may be trained to generate and present a query using syntax, verbs, nouns, and other grammatical elements to provide information and request information as a human being would. The chatbot may be trained to collect information based on an input dataset, such as the exercise information discussed herein. In some embodiments, the chatbot analyzes the input dataset, identify initial patterns in the input dataset, and request additional information to complete the pattern analysis. In some embodiments, the chatbot receives the pattern analysis from a different ML model, such as an exercise analysis model, health habit model, or other ML model. The chatbot may be interactive. For example, the chatbot may be trained to analyze the received response and generate additional content to provide the user. Such content may include recommendations, additional queries, motivational information, any other content, and combinations thereof.

As used herein, a recommendation model may refer to a foundation model that is trained to generate training program recommendations based on an input dataset. The input dataset may include exercise information and/or historical exercise information. Historical exercise information may include any exercise information previously collected. In some embodiments, historical exercise information includes exercise information related to exercise activities prior to the most recent exercise activities. In some embodiments, historical exercise information includes daily exercise information for a period of time (e.g., one day, one week, one month, one year, multiple years). The recommendation model may be trained on a recommendation training dataset. The recommendation training dataset may include exercise information from people and/or prior users that exhibit positive exercise habits and/or have met previously set exercise goals or health habit goals. The recommendation model may receive the exercise information for a user and compare the user's exercise information, exercise goals, and health habit goals to the patterns identified by the recommendation model. The recommendation model may provide behavioral changes for the user to implement to meet his or her goals.

As used herein, the term “health habits” may refer to actions that are consistently performed that result in outcomes associated with positive health. Health habits may refer to any type of behavior, such as sleep-related actions, exercise activity-related actions, food and meal-related actions, any other health actions, and combinations thereof. In some embodiments, health habits are different for different people. In some embodiments, the recommendation model identifies commonalities in the exercise information and health habits, and provide recommendations to change the health habits.

As used herein, an exercise recommendation may be used to refer to any recommendation to improve a user's health, including a recommendation to improve health habits and/or exercise consistency by implementing a training program. For example, the exercise recommendation may include a change in behavior that may be associated with a user's health habits.

As used herein, a training program may include a series of one or more training activities. The training activities may be any type of activity related to a user's health. For example, training activities may include exercise activities, exercise programs, exercises, stretches, health activities, meals, snacks, dietary guidelines, any other training activities, and combinations thereof. In some embodiments, a training program includes certain training activities that are to be performed in a particular order (e.g., the training activities are arranged in a training program order). In some embodiments, the training program includes training activities that are to be performed in a particular time. In some embodiments, the training program includes training activities that are to be performed within a particular timeframe or within a proximity with another training activity. In some embodiments, the training program includes training activities designed to generate and/or improve a user's health habits.

As discussed herein, a training program recommendation system may provide exercise program recommendations to a user using an ML personalization model applied to exercise programs that have been vectorized by a vector search model.

The training program recommendation system may include one or more exercise devices. The exercise devices may include any type of exercise device, such as a treadmill, an elliptical device, a stationary bicycle, a rower, a cable extension device, a mirror including a backlit display behind a mirrored surface, any other exercise device, and combinations thereof. While specific exercise devices are illustrated and discussed herein, it should be understood that the techniques of the present disclosure may be applied to any exercise device that is capable of implementing an exercise program.

The training program recommendation system may further include an exercise program library. The exercise program library may include a repository of one or more exercise programs, assessment programs, training programs, training activities, and other exercise-related information or health-related information. Various elements from the exercise program library may be implemented on one or more of the exercise devices, such as the exercise programs, the assessment programs, the training programs, the training activities, and combinations thereof.

When the user accesses the exercise program (or other activity from the exercise program library) on an exercise device, the exercise program may guide the user through a workout. Throughout the workout, the exercise device controls may adjust one or more operating parameters of the exercise device of an operating feature of the exercise device. The exercise device controls may adjust any operating parameter of the operating feature, such as a flywheel resistance, a belt speed, a device incline, any other operating parameter, and combinations thereof. In some embodiments, the exercise device controls include a duration for one or more portions of the exercise program. For example, the exercise device controls may include a total workout duration for the entire exercise program and/or an interval duration for a period of a particular difficulty level (e.g., resistance level, belt speed, incline level).

In some embodiments, the training program recommendation system collects exercise information from other sources. For example, the training program recommendation system may collect exercise information from one or more wearable devices, such as a smartwatch, a smart ring, a sleep monitor, a heartrate monitor, any other type of wearable device, and combinations thereof. In some examples, the training program recommendation system may collect the exercise information from a computing device, such as a mobile device, a smartphone, a tablet, a laptop computer, a desktop computer, a server computer, any other computing device, and combinations thereof. In some embodiments, the training program recommendation system may collect exercise information using any other type of user device, such as medical devices, GPS trackers, pedometers, any other user devices, and combinations thereof.

The exercise recommendation system may share the exercise information with one or more ML models, such as a health assessment model, a health habit model, a recommendation model, and an exercise chatbot. The ML models may analyze the exercise information to provide recommendations to the user. For example, the health assessment model may analyze the exercise information and identify one or more health parameters for the user. To identify the health parameters, the health assessment model may analyze the exercise information and determine patterns in the user's behavior, including exercise performance (e.g., strength, speed, coordination), health habits, diet information, any other health parameter, and combinations thereof.

The exercise recommendation system may apply the recommendation model to generate training program recommendations. For example, the recommendation model may receive the exercise information, the health parameter and other information to prepare or generate one or more recommendations for the user to implement or change a behavior to improve the user's health and/or exercise consistency. This may help the user to make and attain his or her health and exercise goals.

In accordance with at least one embodiment of the present disclosure, the exercise chatbot may provide the user with an assessment query. The assessment query may request exercise information from the user. In some embodiments, the assessment query requests other information from the user, including exercise goals, weight loss goals, health goals, interests, experience, work, lifestyle, activity level, any other information, and combinations thereof.

In some embodiments, the exercise chatbot enters into a conversation with the user. For example, the exercise chatbot may receive the response including the additional exercise information from the user. The health assessment model and/or the recommendation model may analyze the received additional exercise information. Based on the analysis of the additional exercise information, the exercise chatbot may generate a follow-up query for the user asking for further exercise information. The follow-up query may be related to the received additional exercise information provided by the user as the response to the first query.

The exercise chatbot may send the follow-up query to the user, and the user may provide follow-up exercise information based on the follow-up query. In this manner, the user may enter into a chat or a conversation with the user. The exercise chatbot may provide any number of follow-up queries to the user, and the user may provide any number of responses. In this manner, the exercise chatbot may receive more exercise information with which the training program recommendation system may prepare training program recommendations.

In some embodiments, the follow-up query includes a response to a question from the user. For example, in the response to one or more of the queries, the user may provide a question to the exercise chatbot. The question may be any type of question, such as a request for additional information, a request for clarification, a health question related to health information, an exercise information question related to a particular piece of exercise information or set of exercise information, an exercise activity question related to a particular exercise activity, an exercise device question related to a particular exercise device, any other request for information, and combinations thereof.

In some embodiments, the follow-up query is processed or generated using natural-language processing. For example, the exercise chatbot may include an ML model trained to generate natural language responses. This may help to improve the responsiveness of the exercise chatbot and/or improve the engagement of the user with the exercise chatbot.

Using the received exercise information, the recommendation model may prepare a training program recommendation for the user. The training program recommendation may include any recommendation. For example, the training program recommendation may include a recommendation that the user implements one of the pre-prepared training programs from the exercise program library. In some examples, the training program recommendation may include a recommendation that the user perform a particular exercise program and/or training activity. In some examples, the training program recommendation may include a request that the user perform an assessment program. In some examples, the training program recommendation may request that the user perform the assessment program when the user has not previously performed an assessment program. In some examples, the training program recommendation may request that the user perform the assessment program when the user has previously completed an assessment program, such as to collect additional assessment information and/or to provide a progress update on the results of the user implementing a training program.

In some embodiments, the health assessment model may utilize exercise information collected from the assessment program(s) to generate the health parameter of the user. For example, the health parameter may include a VO2 max. The health assessment model may calculate the VO2 max using exercise information collected during execution of the assessment program. In some embodiments, the health parameter may include any other health parameter, such as average pace, weights lifted, repetitions of an activity, speed, any other health parameter, and combinations thereof.

In some embodiments, the recommendation model may generate the training program recommendation based on the health parameter and/or based on the exercise information collected during the assessment program. In some embodiments, the recommendation model may generate the training program recommendation using historical exercise information and/or health information. In some embodiments, the recommendation model may generate the training program recommendation using the responses to the assessment query asked by the exercise chatbot.

In some embodiments, the recommendation model may generate a training program recommendation that the user implement one or more of the training programs stored in the exercise program library. In some embodiments, the recommendation model may generate a new training program recommendation using one or more of the exercise programs, the training activities, the assessment program, portions of the training programs, complete training programs included within the new training program recommendation, exercise activities, any other activity, and combinations thereof. The newly generated training program may be tailored to the user. This may help to improve the user's exercise experience.

In some embodiments, the recommendation model may prepare an adjusted or an amended training program recommendation while the user is implementing a training program (including while the user is implementing a training program previously recommended by the training program recommendation system). For example, the recommendation model may receive exercise information from the user when the user performs a training activity that is part of the training program. The health assessment model may assess whether the user's health parameter has changed. If the user's health parameter has changed, then the recommendation model may generate an adjusted training program recommendation. For example, if the user's VO2 max has increased, then the recommendation model may provide a recommendation for an increased intensity of the activities included in the training program.

In some embodiments, the health assessment model may determine that the user's exercise status or health parameter have been changed based on the user's performance of a mid-training program assessment program. In some embodiments, the mid-training program assessment program is included as part of the training program. In some embodiments, the mid-training program assessment program is performed by the user when the user thinks he or she has improved their exercise parameter. In some embodiments, the mid-training program assessment program is performed by the user when a trainer provides the recommendation. In some embodiments, the mid-training program assessment program is performed by the user when the health assessment model determines that the user's health parameter has changed.

In accordance with at least one embodiment of the present disclosure, the training program recommendation system may generate a scaling factor for the user. The scaling factor may be used to scale an intensity of activities in the exercise program performed by the user. For example, an exercise programs included in the training program may be performed by and/or based on a performance by a professional athlete. Many users may not be able to perform at the same level of intensity (e.g., strength level, speed, pace, weight setting, resistance setting, number of repetitions, exercise form (e.g., motions)) as the professional athlete. The training program recommendation system may generate a user scaling factor for the user that may adjust the exercise program to the user's intensity level based on the user scaling factor. In this manner, the user may be able to perform the same or similar workouts as the professional athlete.

The health assessment model may identify a user intensity based on the assessment program and/or the user's exercise information. The user intensity may be based on any health parameter. For example, the user intensity may be based on VO2 max, speed, pace, strength, experience, exercise activity form, any other health parameter, and combinations thereof.

Based, at least in part, on the user intensity, the health assessment model and/or the recommendation model may generate the user scaling factor. The user scaling factor may be any type of factor. For example, the user scaling factor may be a percentage or a ratio of the user's anticipated performance compared to a baseline intensity. The exercise programs and/or the training activities may have an activity performance level that is compared to the same baseline intensity. Based on the difference between the user scaling factor and the activity performance level, the exercise program may be scaled to the appropriate level of intensity for the user. In some embodiments, the baseline intensity is different for at least two of the exercise activities.

The scaling factor may scale different activities differently. For example, different trainers may perform the same activity at different levels. To maintain the appropriate level of intensity for the user, the user scaling factor may scale the same activity to the same level for the user, regardless of the trainer or the initial activity level. This may help to maintain a seamless transition between activities for the user.

Each of the components of a training program recommendation system can include software, hardware, or both. For example, the components can include one or more instructions stored on a computer-readable storage medium and executable by processors of one or more computing devices, such as a client device or server device. When executed by the one or more processors, the computer-executable instructions of the training program recommendation system can cause the computing device(s) to perform the methods described herein. Alternatively, the components can include hardware, such as a special-purpose processing device to perform a certain function or group of functions. Alternatively, the components of the training program recommendation system can include a combination of computer-executable instructions and hardware.

Furthermore, the components of the training program recommendation system may, for example, be implemented as one or more operating systems, as one or more stand-alone applications, as one or more modules of an application, as one or more plug-ins, as one or more library functions or functions that may be called by other applications, and/or as a cloud-computing model. Thus, the components may be implemented as a stand-alone application, such as a desktop or mobile application. Furthermore, the components may be implemented as one or more web-based applications hosted on a remote server. The components may also be implemented in a suite of mobile device applications or “apps.”

The exercise recommendation system may include one or more user devices. The user may interact with the user devices to communicate with the exercise recommendation system. For example, the user may input exercise information into the user devices. In some examples, the user may use the user devices to collect exercise information. The user devices may include one or more sensors, and the sensors on the user devices may collect exercise information about the user. As discussed herein, the user devices may include any type of user device, including exercise devices, wearable devices, computing devices, any other user device, and combinations thereof.

The exercise recommendation system may further include an exercise chatbot. The exercise chatbot may communicate with the user regarding exercise information. For example, the exercise chatbot may present the user with queries. The user may respond to the queries to the exercise chatbot. As discussed herein, the exercise chatbot may provide multiple queries to the user and the user may respond to the multiple queries. In his manner the exercise chatbot may hold a conversation or other back-and-forth engagement with the user.

The exercise chatbot may include a query generator. The query generator may be trained to generate queries for the user. For example, the query generator may be trained in natural-language processing to provide queries to the user in a manner that the user may easily understand and be comfortable engaging in. In some examples, the query generator may be trained on exercise information, as discussed herein.

The exercise chatbot may further include an exercise information analyzer. The exercise information analyzer may analyze exercise information, including exercise information received by the user. The exercise information analyzer may communicate with the query generator to prepare the queries.

The training program recommendation system may include a health monitor. The health monitor may receive the exercise information for the user, including exercise information from the user devices and/or the exercise chatbot. In some embodiments, the health monitor receives the exercise information collected from the user during the health assessment activity. The health monitor may collect and organize the exercise information and/or health information from the user.

The training program recommendation system may further include a health assessment model. The health assessment model may analyze the exercise information to generate one or more health parameters for the user. In some embodiments, the health assessment model may generate a user scaling factor for the user.

The exercise recommendation system may further include a recommendation model. The exercise recommendation system may apply the recommendation model to the exercise information. For example, the recommendation model may receive the exercise information from the user devices and/or additional exercise information from the exercise chatbot. In some embodiments, the recommendation model receives exercise analyses from the health assessment model. The recommendation model may be trained to generate a training program recommendation for the user. The recommendation model may provide the exercise recommendation to the user devices. In some examples, the recommendation model may provide the training program recommendation to the exercise chatbot and the exercise chatbot may provide the training program recommendation to the user as a query and/or as part of a conversation with the user.

As discussed herein, the recommendation model may generate the training program recommendation for the user. The training program recommendation may include any training program, including a sequence of exercise activities, training activities, dietary guidelines, health habits, any other activity, and combinations thereof.

In some embodiments, a user performs an assessment on a user device. The assessment may be any type of assessment, such as an assessment exercise activity including pre-determined assessment exercise activities, conventional exercise activities from which assessment exercise information may be obtained, an assessment quiz, any other assessment information, and combinations thereof.

In some embodiments, the user sends, from the user device, a request for an exercise training program. In some embodiments, the request is a user-initiated request. For example, the user may provide an input requesting an exercise training program. In some embodiments, the request is a system-initiated request. For example, the training program recommendation system may identify that the user has performed a particular exercise activity and/or a particular type of exercise activity. The training program recommendation system may generate the request based on the previously performed exercise activity. In some examples, the training program recommendation system may generate the request based on other information, such as the user's exercise information, the user's social media, the user's messages to others, any other information, and combinations thereof.

In some embodiments, an exercise chatbot may receive exercise information from the user. For example, the exercise chatbot may receive exercise information from the assessment, the request, any other exercise information about the user, and combinations thereof. The exercise chatbot may generate an assessment query. The assessment query may include a request for additional information from the user. The assessment query may include a request for any type of information. For example, the assessment query may include a request for the user's goals, fitness level, health habits, diet, interests, any other user information, and combinations thereof.

The exercise chatbot may send the assessment query to the user. For example, the exercise chatbot may send the assessment query to the user device. The user may receive the assessment query at the user device and generate a user response. The user response may include information that is responsive to the assessment query. For example, the user response may include exercise information, goal information, health information, diet information, interests, fitness level, any other user information, and combinations thereof.

As discussed herein, the exercise chatbot may enter into a conversation with the user. For example, the exercise chatbot may receive the user response, including the user information in the user response, and prepare additional assessment queries or follow-up assessment queries. The additional assessment queries may include any request. For example, an additional assessment query may include a request for additional information. In some examples, an additional assessment query may include a request for clarification of the user response. In some examples, the additional assessment query may be part of a series of queries the exercise chatbot may utilize to request information from the user. The user may provide additional user responses to the additional assessment queries. This process may be repeated until the exercise chatbot has requested all the desired user information and/or until the user has provided all the requested user information.

The exercise chatbot may provide the received user information to other elements of the training program recommendation system. For example, the exercise chatbot may provide the user information to a health assessment model and/or a recommendation model. In some examples, the exercise chatbot may instruct the user device to provide the user information to the health assessment model and the recommendation model.

The health assessment model may receive the user information and prepare health a health parameter for the user. As discussed herein, the health parameter may be any health parameter used to assess a user's health. For example, the health parameter may include the users VO2 max. The health assessment model may further prepare a user scaling factor. The user scaling factor may be a representation of the user's exercise ability and/or intensity. In some embodiments, the user scaling factor is normalized to a particular normalizing scale. For example, the user scaling factor may be normalized to a particular exercise parameter, such as speed, pace, strength, resistance level, any other exercise parameter, and combinations thereof. In some examples, the user scaling factor may be normalized to a particular intensity level. In some examples, the user scaling factor may be normalized to a trainer's fitness level. For example, the user's scaling factor may be normalized based on a ratio between the user's VO2 max and a trainer VO2 max of a trainer in the training program.

In some embodiments, the health assessment model prepares a single user scaling factor for the user. The single user scaling factor may be used to scale each of the exercise activities in a particular training plan. In some embodiments, the health assessment model prepares multiple user scaling factors for the user. In this manner, at least two of the exercise activities may have different user scaling factors. For example, the user may have different fitness or intensity levels for different activities. As a specific, non-limiting example, a user may have a relatively high intensity level for running and a relatively low intensity level for weight lifting. The health assessment model may prepare different scaling factors for different activities and/or activity types. This may help to adjust each exercise activity to the user's ability and/or intensity, thereby improving the user experience.

The health assessment model may send the health parameter and scaling factor to the recommendation model. The recommendation model may utilize the health parameter, the scaling factor, and the user information to prepare one or more training program recommendations. For example, the recommendation model may prepare a recommendation of a pre-existing training program. In some examples, the recommendation model may prepare a recommendation for a new training program using pre-existing exercise activities and other training activities.

The recommendation model may send the training program recommendations to the user. In some embodiments, the recommendation model sends a single training program recommendation to the user. This may help to reduce the user's confusion and stress in selecting a training program recommendation. In some embodiments, the recommendation model sends multiple training program recommendations to the user. This may allow the user to select which training program he or she favors.

In accordance with at least one embodiment of the present disclosure, when the user selects the training program, the user device may implement the training program. For example, the user device may implement, in the sequence of the training program, the training activities from the training program. In some embodiments, a particular user device may implement the training activities that are capable of being implement on the user device. For example, a treadmill may implement training activities involving walking or running. In some embodiments, the user device includes multiple user devices, with each user device implementing training activities that are capable of being implemented on the user device. In this manner, the training program may help to improve the user's health and/or compliance with a particular training program.

The following discussion and the examples provide a number of different methods, systems, devices, and computer-readable media of the training program recommendation system discussed herein. The methods discussed herein may be performed with more or fewer acts. Further, the acts may be performed in differing orders. Additionally, the acts described herein may be repeated or performed in parallel with one another or parallel with different instances of the same or similar acts.

In accordance with at least one embodiment of the present disclosure, the training program recommendation system may monitor a user while the user is performing an assessment exercise activity. As discussed herein, the assessment exercise activity may be any exercise activity, including an exercise specifically directed to assessing the user's fitness or intensity level, a previously performed exercise activity, a regularly scheduled exercise activity, any other exercise activity, and combinations thereof.

In some embodiments, the training program recommendation system presents, with an exercise chatbot, the user with an assessment query. The training program recommendation system may apply a health assessment model to the exercise information to generate a health parameter. The health parameter may be based, at least in part, on the assessment exercise activity. In some embodiments, the training program recommendation system may determine a user scaling factor based on the assessment exercise activity and/or the user's responses to the assessment query.

The training program recommendation system may apply a recommendation model to the health parameter and the user's response to the assessment query. The recommendation model may generate a training program recommendation for the user. In some embodiments, the training program recommendation is based on the health parameter and/or the user scaling factor. The training program recommendation may include a training program. The training program recommendation system may provide the training program to the user.

The training program recommendation system may monitor a user while performing an exercise activity. As discussed herein, the exercise activity may be any exercise activity, including an assessment exercise activity and/or an exercise activity performed without the intent to receive an assessment. The training program recommendation system may determine a health parameter for the user based on the exercise activity. The training program recommendation system may receive exercise information for the user.

The training program recommendation system may apply a health assessment model to the exercise information and the health parameter. The heal assessment model may identify a user scaling factor for the user. The training program recommendation system may present an assessment query to the user. The assessment query may request user information. The requested user information may include user goals. The training program recommendation system may apply a recommendation model to the user scaling factor and a response from the user. The response from the user may include the user goals. The recommendation model may generate a training program for the user. The training program may be scaled based on the user scaling factor.

One or more computer systems may be used to implement the various devices, components, and systems described herein. The computer system includes a processor. The processor may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor may be referred to as a central processing unit (CPU). Although just a single processor is described herein, in an alternative configuration, a combination of processors (e.g., an ARM and DSP) could be used.

The computer system also includes memory in electronic communication with the processor. The memory may be any electronic component capable of storing electronic information. For example, the memory may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions and data may be stored in the memory. The instructions may be executable by the processor to implement some or all of the functionality disclosed herein. Executing the instructions may involve the use of the data that is stored in the memory. Any of the various examples of modules and components described herein may be implemented, partially or wholly, as instructions stored in memory and executed by the processor. Any of the various examples of data described herein may be among the data that is stored in memory and used during execution of the instructions by the processor.

A computer system may also include one or more communication interfaces for communicating with other electronic devices. The communication interface(s) may be based on wired communication technology, wireless communication technology, or both. Some examples of communication interfaces include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system may also include one or more input devices and one or more output devices. Some examples of input devices include a keyboard, mouse, microphone, remote control device, button, joystick, trackball, touchpad, and lightpen. Some examples of output devices include a speaker and a printer. One specific type of output device that is typically included in a computer system is a display device. Display devices used with embodiments disclosed herein may utilize any suitable image projection technology, such as liquid crystal display (LCD), light-emitting diode (LED), gas plasma, electroluminescence, or the like. A display controller may also be provided, for converting data stored in the memory into text, graphics, and/or moving images (as appropriate) shown on the display device.

The various components of the computer system may be coupled together by one or more buses, which may include a power bus, a control signal bus, a status signal bus, a data bus, etc.

Following are sections in accordance with at least one embodiment of the present disclosure:
A1. A method for providing training program recommendations, comprising:
monitoring a user while the user is performing an assessment exercise activity;
presenting, with an exercise chatbot, the user with an assessment query, the assessment query requesting exercise information for the user;
applying a health assessment model to the exercise information to determine a health parameter and a user scaling factor, the health parameter based at least in part on the assessment exercise activity;
applying a recommendation model to the health parameter and a user response to the assessment query, the recommendation model generating a training program recommendation for the user based at least in part on at least one of the health parameter or the user scaling factor, the training program recommendation including a training program; and providing the training program recommendation to the user.
A2. The method of section A1, further comprising implementing an exercise activity associated with the training program.
A3. The method of section A2, wherein implementing the exercise activity includes implementing the exercise activity on an exercise device.
A4. The method of any of sections A2 or A3, wherein implementing the exercise activity includes scaling an intensity of the exercise activity based on the user scaling factor.
A5. The method of any of sections A1-A4, wherein the training program includes a plurality of exercise activities.
A6. The method of section A5, wherein the plurality of exercise activities are arranged in a training program order of the training program.
A7. The method of any of sections A5 or A6, wherein the user scaling factor is different for at least two of the plurality of exercise activities.
A8. The method of any of sections A1-A7, wherein the user scaling factor is based on the health parameter and a baseline intensity of the training program.
A9. The method of section A8, wherein the training program includes a plurality of exercise activities, and wherein the baseline intensity is different for at least two of the plurality of exercise activities.
A10. The method of any of sections A1-A9, wherein the health parameter includes a VO2 max.
A11. The method of section A10, wherein the user scaling factor is based on a ratio between the VO2 max and a trainer VO2 max of a trainer in the training program.
A12. The method of any of sections A1-A11, wherein the exercise information includes user exercise goals.
A13. The method of any of sections A1-A12, wherein the exercise information includes gender.
A14. The method of any of sections A1-A13, wherein the exercise information includes age.
A15. The method of any of sections A1-A14, wherein the training program includes a plurality of training programs, and wherein presenting the training program recommendation includes presenting the plurality of training programs to the user.
A16. The method of section A15, wherein the plurality of training programs include a training activity directed to user exercise goals of the user.
A17. The method of any of sections A1-A16, wherein the assessment exercise activity is implemented on an exercise device.
A18. The method of section A17, wherein the exercise device includes a treadmill.
A19. The method of any of sections A17 or A18, wherein the exercise device includes an elliptical device.
A20. The method of any of sections A17-A19, wherein the exercise device includes a rower.
A21. The method of any of sections A17-A20, wherein the exercise device includes a cable extension device.
A22. The method of any of sections A1-A21, wherein monitoring the user includes collecting heartrate information.
A23. The method of any of sections A1-A22, wherein monitoring the user includes collecting blood oxygen levels.
A24. The method of any of sections A1-A23, wherein monitoring the user includes collecting video of the user.
A25. The method of any of sections A1-A24, wherein preparing the training program recommendation includes generating the training program recommendation.
A26. The method of section A25, wherein generating the training program includes arranging a plurality of exercise activities based on the health parameter and the user scaling factor.
A27. The method of any of sections A1-A26, wherein the assessment exercise activity is a regularly scheduled exercise activity.
B1. A method for providing training program recommendations, the method comprising:
monitoring a user while performing an exercise activity;
determining a health parameter for the user based on the exercise activity;
receiving exercise information for the user;
applying a health assessment model to the exercise information and the health parameter, the health assessment model identifying a user scaling factor for the user;
presenting a query to the user, the query requesting user information including user goals;
applying a recommendation model to the user scaling factor and a response from the user including the user goals, the recommendation model generating a training program for the user.
B2. The method of section B1, wherein monitoring the user includes monitoring the user while performing the exercise activity on an exercise device.
B3. The method of any of sections B1 or B2, further comprising implementing an exercise activity associated with the training program.
B4. The method of section B3, wherein implementing the exercise activity includes implementing the exercise activity on an exercise device.
B5. The method of any of sections B1-B4, further comprising scaling the training program based on the user scaling factor.
B6 The method of any of sections B1-B5, wherein the recommendation model is trained on exercise activities implemented by trainers and prior users.
B7. The method of any of sections B1-B6, further comprising generating the query using an exercise chatbot.
B8. The method of section B7, wherein the exercise chatbot includes a large language model trained in natural language algorithms.
B9. The method of any of sections B1-B8, wherein the exercise information includes prior workouts performed.
B10. The method of any of sections B1-B9, wherein the exercise information includes user exercise program ratings.
B11. The method of any of sections B1-B10, wherein the exercise information includes historical exercise information.
B12. The method of any of sections B1-B11, wherein the exercise information includes an exercise activity type.
B13. The method of any of sections B1-B12, wherein the exercise information includes historical training plan information.
B14. The method of any of sections B1-B13, wherein the health parameter includes heartrate.
B15. The method of any of sections B1-B14, wherein the health parameter includes a VO2 max.
B16. The method of section B15, wherein the exercise activity is an assessment exercise activity configured to calculate the VO2 max.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for providing training program recommendations, comprising:
monitoring a user while the user is performing an assessment exercise activity;
presenting, with an exercise chatbot, the user with an assessment query, the assessment query requesting exercise information for the user;
applying a health assessment model to the exercise information to determine a health parameter and a user scaling factor, the health parameter based at least in part on the assessment exercise activity;
applying a recommendation model to the health parameter and a user response to the assessment query, the recommendation model generating a training program recommendation for the user based at least in part on at least one of the health parameter or the user scaling factor, the training program recommendation including a training program; and
providing the training program recommendation to the user.

2. The method of claim 1, further comprising:
implementing an exercise activity associated with the training program.

3. The method of claim 2, wherein implementing the exercise activity comprises:
implementing the exercise activity on an exercise device.

4. The method of claim 2, wherein implementing the exercise activity comprises:
scaling an intensity of the exercise activity based on the user scaling factor.

5. The method of claim 1, wherein the training program comprises a plurality of exercise activities.

6. The method of claim 5, wherein the plurality of exercise activities are arranged in a training program order of the training program.

7. The method of claim 5, wherein the user scaling factor is different for at least two of the plurality of exercise activities.

8. The method of claim 1, wherein the user scaling factor is based at least in part on the health parameter and a baseline intensity of the training program.

9. The method of claim 8, wherein the training program comprises a plurality of exercise activities, and wherein the baseline intensity is different for at least two of the plurality of exercise activities.

10. A method for providing training program recommendations, comprising:
monitoring a user while performing an exercise activity;
determining a health parameter for the user based at least in part on the exercise activity;
receiving exercise information for the user;
applying a health assessment model to the exercise information and the health parameter, the health assessment model identifying a user scaling factor for the user;
presenting a query to the user, the query requesting user information including user goals; and
applying a recommendation model to the user scaling factor and a response from the user including the user goals, the recommendation model generating a training program for the user.

11. The method of claim 10, wherein monitoring the user comprises:
monitoring the user while performing the exercise activity on an exercise device.

12. The method of claim 10, further comprising:
implementing an exercise activity associated with the training program.

13. The method of claim 12, wherein implementing the exercise activity comprises:
implementing the exercise activity on an exercise device.

14. The method of claim 10, further comprising:
scaling the training program based on the user scaling factor.

15. The method of claim 10, further comprising:
generating the query using an exercise chatbot.

16. The method of claim 10, wherein the health parameter includes a VO2 max.

17. The method of claim 16, wherein the exercise activity is an assessment exercise activity configured to calculate the VO2 max.

18. An exercise system comprising:
an exercise device; and
one or more processors and one or more memories, the one or more memories comprising instructions that, when executed by the one or more processors, cause the exercise system to:
monitor a user while the user is performing an assessment exercise activity;
present, with an exercise chatbot, the user with an assessment query, the assessment query requesting exercise information for the user;
apply a health assessment model to the exercise information to determine a health parameter and a user scaling factor, the health parameter based at least in part on the assessment exercise activity;
apply a recommendation model to the health parameter and a user response to the assessment query, the recommendation model generating a training program recommendation for the user based at least in part on at least one of the health parameter or the user scaling factor, the training program recommendation including a training program; and
provide the training program recommendation to the user.

19. The exercise system of claim 18, wherein the instructions, when executed by the one or more processors, further cause the exercise system to:
implement the training program on the exercise device.

20. The exercise system of claim 19, wherein the instructions to implement the training program, when executed by the one or more processors, cause the exercise system to:
scale an intensity of the training program based at least in part on the user scaling factor.
